(19) United States
(12) Patent Application Publication
(10) Pub. No.: US 2005/0176792 A1
(43) Pub. Date: Aug. 11, 2005

(54) Ketone substituted benzimidazole compounds

(75) Inventors: Kevin J. Moriarty, East Norriton, PA (US); Lei Qiao, Dowington, PA (US); Gregory Paul Roth, Woodstock, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/035,876

(22) Filed: Jan. 7, 2005

Related U.S. Application Data
(60) Provisional application No. 60/536,362, filed on Jan. 14, 2004.

(51) Int. Cl. A61K 31/42; A61K 31/4184; C07D 413/02; C07D 45/02

(52) U.S. Cl. 514/379; 514/394; 548/248; 548/304.7

(57) ABSTRACT

Disclosed are ketone substituted benzimidazole compounds of formula(I): 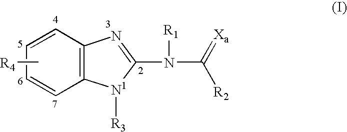
wherein R₁, R₂, R₃, R₄ and Xₐ are defined herein. The compounds of the invention inhibit Itk kinase and are therefore useful for treating diseases and pathological conditions involving inflammation, immunological disorders and allergic disorders. Also disclosed are processes for preparing these compounds and to pharmaceutical compositions comprising these compounds.

### APPLICATION DATA

This application claims benefit to U.S. provisional application no. 60/536,362 filed Jan. 14, 2004.

### TECHNICAL FIELD OF THE INVENTION

This invention relates to substituted benzimidazole compounds of formula(I): 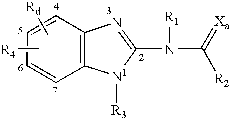
wherein R₁, R₂, R₃, R₄ and Xₐ are defined herein below. The compounds of the invention inhibit Itk kinase and are therefore useful for treating diseases and pathological conditions involving inflammation, immunological disorders and allergic disorders. This invention also relates to processes for preparing these compounds and to pharmaceutical compositions comprising these compounds.

### BACKGROUND OF THE INVENTION

Protein kinases play a critical role in mediating signaling events leading to cellular responses such as activation, growth and differentiation, in response to extracellular signals. Protein kinases transmit their signal by phosphorylating specific residues in a target protein. Protein kinases that specifically phosphorylate tyrosine residues are referred to as protein tyrosine kinases. Protein tyrosine kinases can be divided into two general groups: receptor such as epidermal growth factor (EGF) receptor (S. Iwashita and M. Kobayashi, 1992, Cellular Signalling, 4, 123-132) and cytosolic non-receptor (C. Chan et al., 1994, Ann. Rev. Immunol., 12, 555-592).

Interleukin-2-inducible T cell kinase (Itk), also referred to as T cell-specific kinase (Tsk) and expressed mainly in T-lymphocytes (EMT), is a member of the Tec family of protein tyrosine kinases that also includes Txk, Tec, Btk, and Bmx. Tec family members are characterized by the presence of a pleckstrin-homology domain (PH), a proline rich Tec homology domain (TH) and Src homology SH3, SH2 and SH1 kinase domains positioned from the N-terminus to the C-terminus respectively (S. Gibson et al., 1993, Blood, 82,1561-1572; J. D. Siliciano et al., 1992, Proc. Nat. Acad. Sci., 89, 11194-11198; N. Yamada et al., 1993 Biochem. and Biophys Res. Comm., 192, 231-240).

Itk is expressed in T cells, mast cells and natural killer cells. It is activated in T cells upon stimulation of the T cell receptor (TCR), and in mast cells upon activation of the high affinity IgE receptor. Following receptor stimulation in T cells, Lck, a src tyrosine kinase family member, phosphorylates Y511 in the kinase domain activation loop of Itk (S. D. Heyeck et al., 1997, J. Biol. Chem, 272, 25401-25408). Activated Itk, together with Zap-70 is required for phosphorylation and activation of PLC-γ (S. C. Bunnell et al., 2000, J. Biol. Chem., 275, 2219-2230). PLC-γ catalyzes the formation of inositol 1,4,5-triphosphate and diacylglycerol, leading to calcium mobilization and PKC activation, respectively. These events activate numerous downstream pathways and lead ultimately to degranulation (mast cells) and cytokine gene expression (T cells) (Y. Kawakami et al., 1999, J. Leukocyte Biol., 65, 286-290).

The role of Itk in T cell activation has been confirmed in Itk knockout mice. CD4⁺T cells from Itk knockout mice have a diminished proliferative response in a mixed lymphocyte reaction or upon Con A or anti-CD3 stimulation. (X. C. Liao and D. R. Littman, 1995, Immunity, 3, 757-769). Also, T cells from Itk knockout mice produced little IL-2 upon TCR stimulation resulting in reduced proliferation of these cells. In another study, Itk deficient CD4⁺T cells produced reduced levels of cytokines including IL-4, IL-5 and IL-13 upon stimulation of the TCR, even after priming with inducing conditions. (D. J. Fowell, 1999, Immunity, 11, 399-409).

The role of Itk in PLC-Y activation and in calcium mobilization was also confirmed in the T cells of these knockout mice, which had severely impaired IP₃ generation and no extracellular calcium influx upon TCR stimulation (K. Liu et al., 1998, J. Exp. Med. 187, 1721-1727). The studies described above support a key role for Itk in activation of T cells and mast cells. Thus an inhibitor of Itk would be of therapeutic benefit in diseases mediated by inappropriate activation of these cells.

It has been well established that T cells play an important role in regulating the immune response (Powrie and Coffman, 1993, Immunology Today, 14, 270-274). Indeed, activation of T cells is often the initiating event in immunological disorders. Following activation of the TCR, there is an influx of calcium that is required for T cell activation. Upon activation, T cells produce cytokines, including IL-2, 4, 5, 9, 10, and 13 leading to T cell proliferation, differentiation, and effector function. Clinical studies with inhibitors of IL-2 have shown that interference with T cell activation and proliferation effectively suppresses immune response in vivo (Waldmann, 1993, Immunology Today, 14, 264-270). Accordingly, agents that inhibit T lymphocyte activation and subsequent cytokine production, are therapeutically useful for selectively suppressing the immune response in a patient in need of such immunosuppression.

Mast cells play a critical roll in asthma and allergic disorders by releasing pro-inflammatory mediators and cytokines. Antigen-mediated aggregation of FcεRI, the high-affinity receptor for IgE results in activation of mast cells (D. B. Corry et al., 1999, Nature, 402, B 18-23). This triggers a series of signaling events resulting in the release of mediators, including histamine, proteases, leukotrienes and cytokines (J. R. Gordon et al., 1990, Immunology Today, 11, 458-464.) These mediators cause increased vascular permeability, mucus production, bronchoconstriction, tissue degradation and inflammation thus playing key roles in the etiology and symptoms of asthma and allergic disorders.

Recent published data using Itk knockout mice suggests that in the absence of Itk function, increased numbers of memory T cells are generated (A. T. Miller et al., 2002 The Journal of Immunology, 168, 2163-2172). One strategy to improve vaccination methods is to increase the number of memory T cells generated (S. M. Kaech et al., Nature Reviews Immunology, 2, 251-262).

All documents cited in this application are incorporated by reference in their entirety.

### SUMMARY OF THE INVENTION

It is therefore an object of the invention to provide a compound of the formula (I): 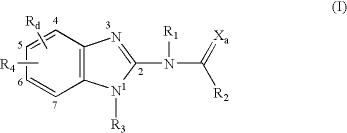
wherein R₁, R₂, R₃, R₄ and Xₐ are defined herein below.

It is another object of the invention to provide a method of inhibiting the Tec kinase family, including Itk kinase, and methods of treating diseases or conditions related to such kinase activity activity, by administering to a patient in need thereof a therapeutically effective amount of a compound of the formula (I).

It is yet another object of the invention to provide pharmaceutical compositions and processes of making compounds of the formula (I) as described herein below.

### DETAILED DESCRIPTION OF THE PREFERRED EMBODIMENTS

In it's broadest generic embodiment, the invention provides for a compound of the formula (I): 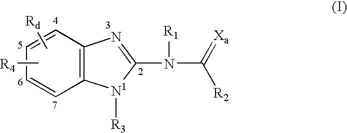
wherein:
R₁ is hydrogen or alkyl;
R₂ is chosen from aryl and heteroaryl each R₂ is optionally substituted with one or more Rₐ;
R₃ is C₁₋₁₀ alkyl chain branched or unbranched optionally substituted with one or more R_{b},
   or R₃ is the group:
      —(CH₂)ₙ-L-R₆, wherein L is chosen from a bond, —NH—C(O)—, —O—C(O)—, —C(O)— and —S(O)ₘ— wherein m is 0, 1 or 2, and wherein said group is optionally substituted by one or more R_{b};
   wherein R₆ is independently chosen from hydrogen, hydroxy, alkyl, alkoxy, alkylthio, arylC₀₋₅ alkyl, aryloxyC₀₋₅ alkyl, heteroarylC₀₋₅ alkyl, cycloalkylC₀₋₅ alkyl, heterocyclylC₀₋₅ alkyl and amino said amino is optionally mono-or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;
   n is 1-10;
R₄ is a group chosen from: 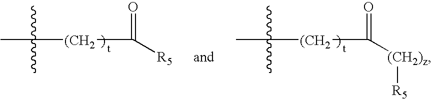
   wherein a hydrogen atom for each of the —(CH₂)— groups may be replaced with a C₁₋₁₀ alkyl wherein one or more —CH₂— groups of said alkyl are optionally replaced by a heteroatom group chosen from O, S and NH, R₄ is covalently attached at the indicated 5- or 6-position of the formula (I), t and z are each independently chosen from 0,1 or 2;
R₅ is chosen from arylC₀₋₅ alkyl, alkyl, heteroarylC₀₋₅ alkyl, cycloalkylC₀₋₅ alkyl and heterocyclylC₀₋₅ alkyl, each R₅ optionally substituted with one or more R_{c};
each Rₐ, R_{b} or R_{c} are independently chosen from hydrogen, alkyl, alkenyl, alkynyl, cycloalkyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above Rₐ, R_{b} or R_{c} are optionally halogenated where possible;
R_{d}, covalently attached at the indicated 4-, 5-, 6- or 7-position of the formula (I), is chosen from hydrogen, alkyl, alkoxy and halogen and
Xₐ and X_{b} are oxygen or sulfur;
or the pharmaceutically acceptable salts, esters, acids, isomers or tautomers thereof.

In another embodiment, there is provided a compound of the formula (I) as described immediately above and wherein:
R₁ is hydrogen;
R₂ is chosen from phenyl, naphthyl, and heteroaryl chosen from thienyl, furanyl, isoxazolyl, oxazolyl, thiazolyl, thiadiazolyl, tetrazolyl, pyrazolyl, pyrrolyl, imidazolyl, pyridinyl; pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl each R₂ is optionally substituted with one or more Rₐ;
R₃ is C₁₋₁₀ alkyl chain branched or unbranched optionally substituted with one or more R_{b},
   or R₃ is:
      —(CH₂)ₙ-L-R₆, wherein L is chosen from a bond, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, C₁₋₅ alkyl, C₁₋₅ alkoxy, C₁₋₅ alkylthio, phenyl, naphthyl, benzyl, phenethyl, heteroarylC₀₋₅ alkyl, C₃₋₇ cycloalkylC₀₋₅ alkyl, heterocyclylC₀₋₅ alkyl and amino said amino is optionally mono-or di-substituted by C₁₋₅ acyl, C₁₋₅ alkyl, C₁₋₅ alkoxycarbonyl, arylC₀₋₅ alkyl, heteroarylC₀₋₅ alkyl or heterocyclylC₀₋₅ alkyl; and wherein each recited heteroaryl in this paragraph is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl and wherein each recited heterocyclyl in this paragraph is chosen from pyrrolidinyl, morpholinyl, thiomorpholinyl, dioxalanyl, piperidinyl and piperazinyl;
R₄ is a group chosen from: 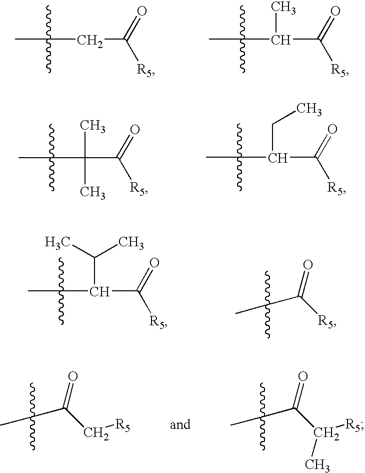
R₅ is chosen from phenyl, naphthyl, benzyl, phenethyl, C₁₋₅ alkyl, heteroarylC₀₋₅ alkyl wherein the heteroaryl is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl, C₃₋₇ cycloalkylC₀₋₅ alkyl and heterocyclylC₀₋₅ alkyl wherein the heterocyclyl is chosen from aziridinyl, pyrrolidinyl, morpholinyl, thiomorpholinyl, tetrahydrofuranyl, dioxalanyl, piperidinyl and piperazinyl, each R₅ is optionally substituted with one or more R_{c};
each Rₐ, R_{b} or R_{c} are independently chosen from hydrogen, C₁₋₅ alkyl, C₂₋₅ alkenyl, C₂₋₅ alkynyl, C₃₋₈ cycloalkyl, phenyl, benzyl, phenoxy, C₁₋₅ alkoxy, C₁₋₅ alkylthio, C₁₋₅ acyl, C₁₋₅ alkoxycarbonyl, C₁₋₅ acyloxy, C₁₋₅ acylamino, C₁₋₅ sulphonylamino, aminosulfonyl, C₁₋₅ alkylsulfonyl, carboxy, carboxamide, oxo, hydroxy, halogen, trifluoromethyl, nitro, nitrile and amino optionally mono-or-di-substituted by C₁₋₅ alkyl, C₁₋₅ acyl or C₁₋₅ alkoxycarbonyl, wherein any of the above Rₐ, R_{b} or R_{c} are optionally halogenated where possible;
R_{d} is chosen from hydrogen, C₁₋₃ alkyl, C₁₋₃ alkoxy and halogen;
and
Xₐ is oxygen.

In yet another embodiment, there is provided a compound of the formula (I) as described immediately above and wherein:
R₂ is chosen from phenyl, naphthyl and heteroaryl chosen from thienyl, furanyl, isoxazolyl, oxazolyl, imidazolyl, thiadiazolyl, pyrazolyl, pyridinyl, quinoxalinyl and benzothienyl each R₂ is optionally substituted with one or more Rₐ;
R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono-or di-substituted by C₁₋₅ acyl, C₁₋₅ alkyl, C₁₋₅ alkoxycarbonyl, arylC₀₋₅ alkyl or heteroarylC₀₋₅ alkyl;
   and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b};
n is 1-6;
R₅ is chosen from phenyl, naphthyl, benzyl, phenethyl, C₁₋₅ alkyl, heteroarylC₀₋₅ alkyl wherein the heteroaryl in this paragraph is chosen from thienyl, furanyl, imidazolyl and pyridinyl, C₃₋₇ cycloalkylC₀₋₅ alkyl and heterocyclylC₀₋₅ alkyl wherein the heterocyclyl is chosen from aziridinyl, pyrrolidinyl, tetrahydrofuranyl, tetrahydropyridinyl, morpholinyl, thiomorpholinyl, piperidinyl and piperazinyl, each R₅ is optionally substituted with one or more R_{c};
R_{d} is chosen from hydrogen and C₁₋₃ alkyl.

In yet still another embodiment, there is provided a compound of the formula (I) as described immediately above and wherein:
R₂ is chosen from phenyl and heteroaryl chosen from thienyl, furanyl, isoxazolyl, thiadiazolyl, pyrazolyl and pyridinyl each R₂ is optionally substituted with one or more Rₐ;
R₃ is:
—(CH₂)ₙ—C(O)—R₆ or
—(CH₂)ₙ—R₆;
   wherein R₆ is independently chosen from hydroxy, C₁₋₅ alkyl, C₁₋₅ alkoxy, phenyl, morpholinylC₀₋₅ alkyl, piperazinylC₀₋₅ alkyl, imidazolylC₀₋₅ alkyl, pyrrolidinylC₀₋₅ alkyl, pyrrolidinonylC₀₋₅ alkyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from phenyl, furanyl, benzyl, phenethyl, C₁₋₃ alkyl and C₃₋₇ cycloalkylC₀₋₅ alkyl each optionally substituted with one or more R_{c};
each Rₐ, R_{b} or R_{c} are independently chosen from C₁₋₅ alkyl, C₃₋₈ cycloalkyl, phenyl, C₁₋₅ alkoxy, amino optionally mono-or-di-substituted by C₁₋₅ alkyl, C₁₋₅ alkoxycarbonyl, carboxamide, hydroxy, halogen, trifluoromethyl, nitro and nitrile, wherein any of the above Rₐ, R_{b} or R_{c} are optionally halogenated where possible;
and
R_{d} is chosen from hydrogen and methyl.

In a further embodiment, there is provided a compound of the formula (I) as described immediately above and wherein:
R₂ is chosen from phenyl, thienyl, furanyl, isoxazolyl and pyridinyl each optionally substituted with one or more Rₐ;
R₅ is chosen from methyl, CF₃, cyclopentyl, phenyl and cyclohexyl each optionally substituted with one or more R_{c};
R_{d} is hydrogen and
n is 2-5.

In yet another embodiment, there is provided a compound of the formula (I) as described immediately above and wherein:
R₂ is chosen from phenyl, thien-2-yl, isoxazol-5-yl and pyridin-3-yl each optionally substituted with one or more Rₐ;
R₄ is chosen from: 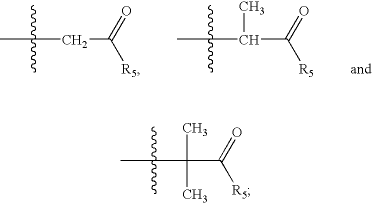
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl;
and
each Rₐ, R_{b} or R_{c} are independently chosen from C₁₋₃ alkoxy, amino optionally mono-or-di-substituted by C₁₋₃ alkyl, carboxamide, hydroxy, fluoro, chloro, bromo, trifluoromethyl, nitro and nitrile.

In any of the aforementioned embodiments, there are provided compounds of the formula (I) wherein:
R₄ is covalently attached at the indicated 5-position of the formula (I) or in another embodiment R₄ is covalently attached at the indicated 6-position of the formula (I).

In another embodiment there is provided representative compounds of the invention which can be made in accordance with the general schemes and working examples presented below: 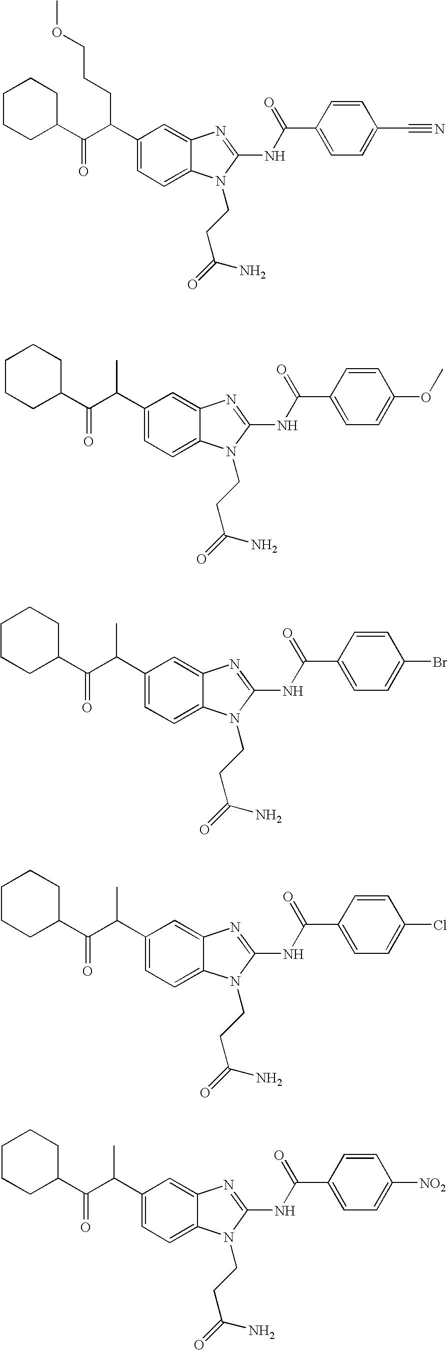 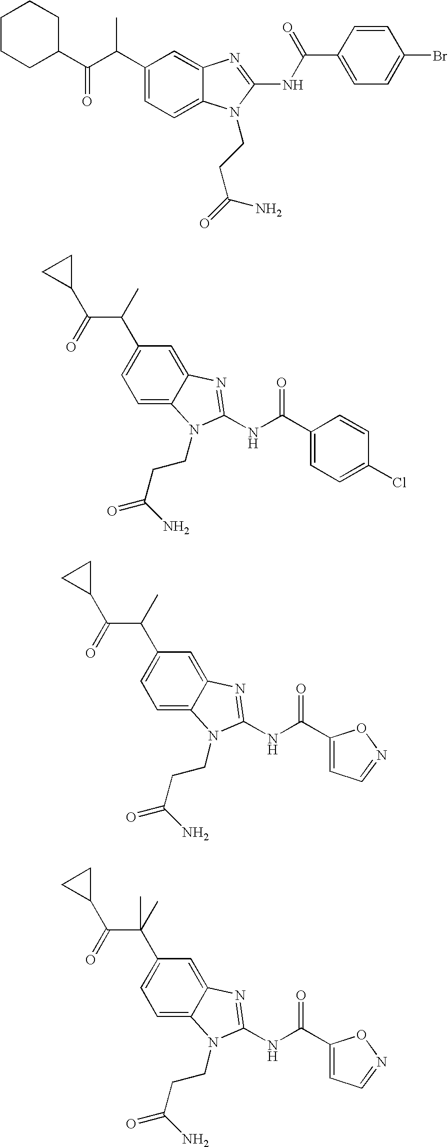 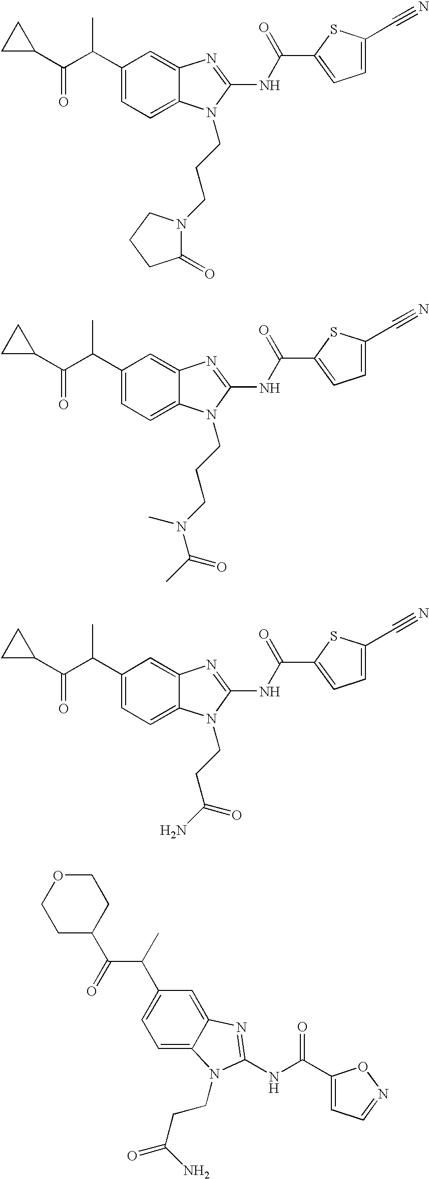 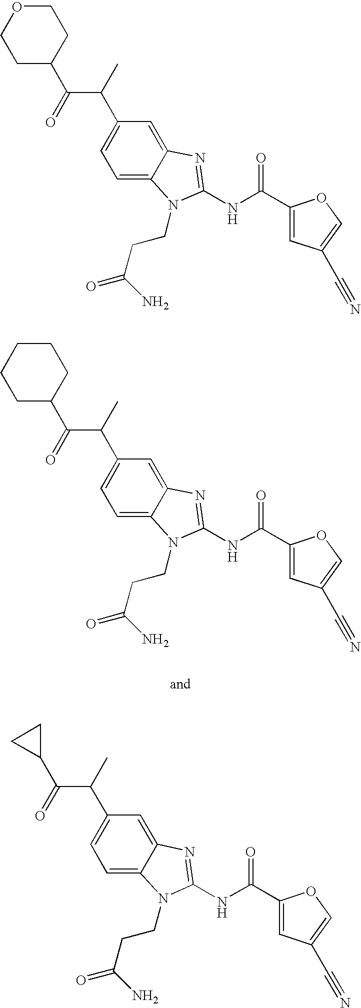
or
the pharmaceutically acceptable salts, esters, acids, isomers or tautomers thereof.

In another embodiment there is provided representative compounds of the invention which can be made in accordance with the general schemes and working examples presented below: 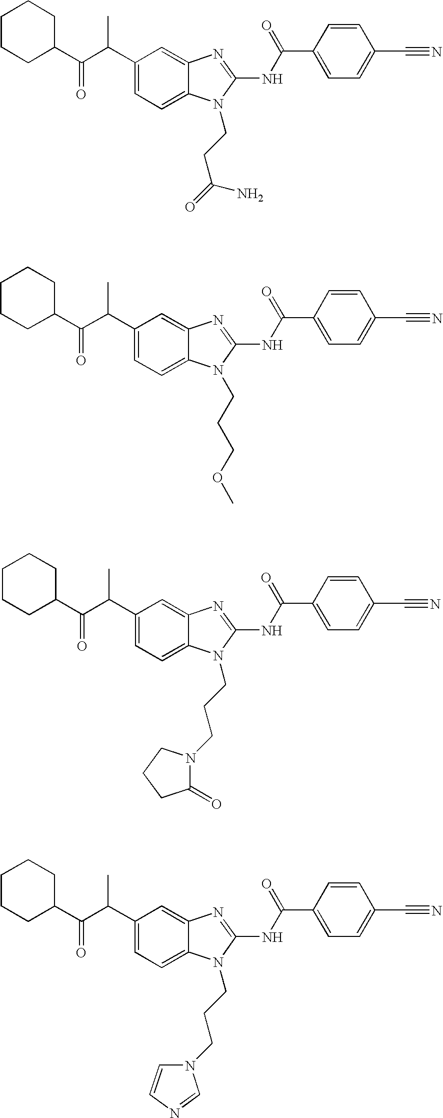 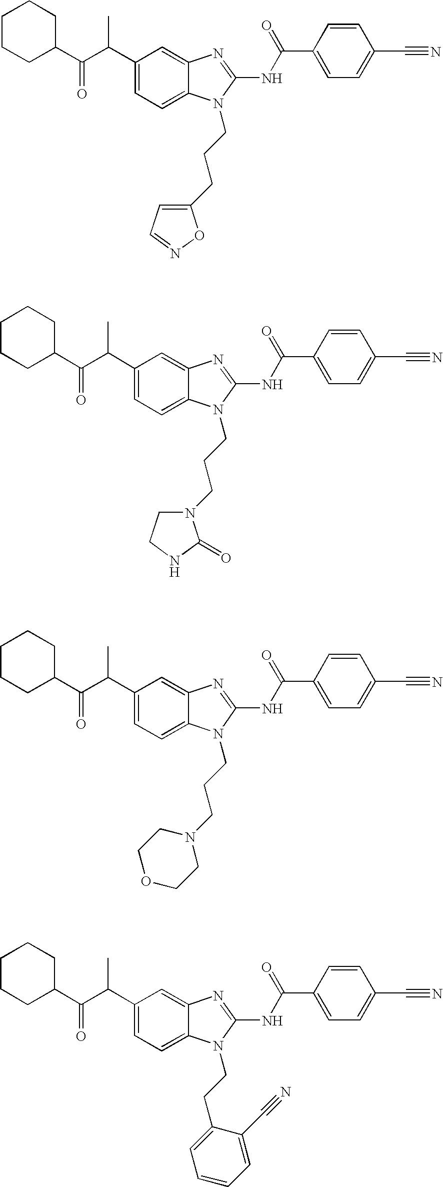 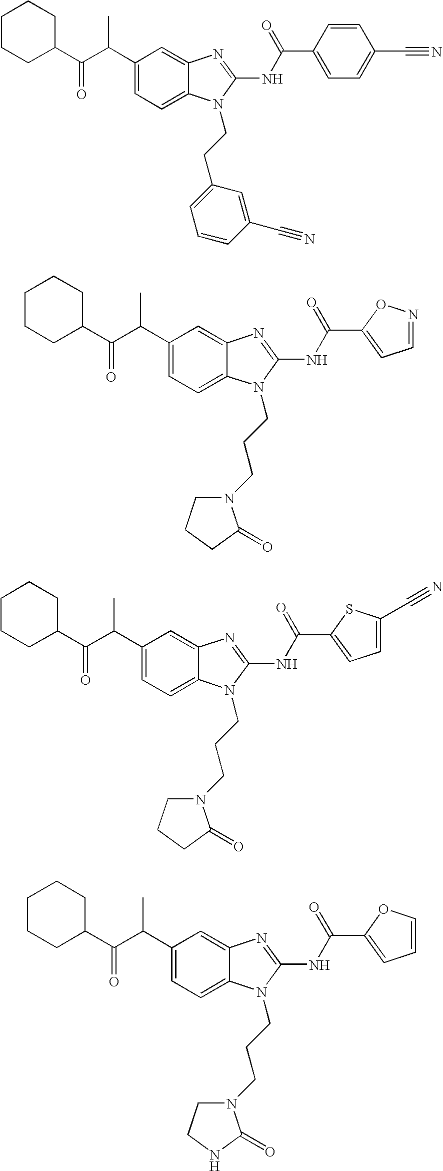 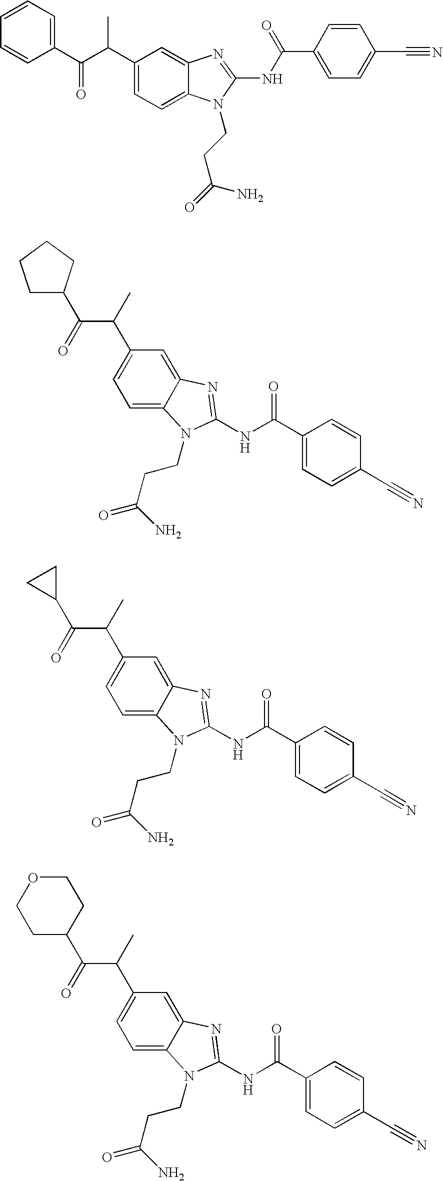 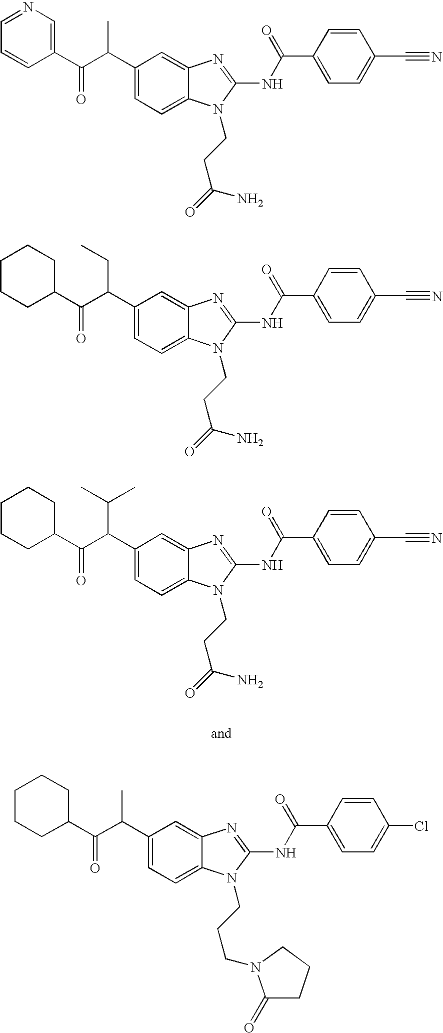
or the pharmaceutically acceptable salts, esters, acids, isomers or tautomers thereof.

Any of the aforementioned embodiments disclosed above may have Rₐ, R_{b} or R_{c} also being defined as azido. Such compounds are useful as photolabeling probes and include, for example, 4-azido-phenyl moieties.

In all the compounds disclosed herein above in this application, in the event the nomenclature is in conflict with the structure, it shall be understood that the compound is defined by the structure.

Of particular importance according to the invention are the abovementioned compounds for use as pharmaceutical compositions with anti-Tec kinase activity.

The invention also relates to compounds as described herein for preparing a pharmaceutical composition for the treatment and/or prevention of a Tec kinase mediated disease or condition.

The invention also relates to pharmaceutical preparations, containing as active substance one or more compounds as described herein, or the pharmaceutically acceptable derivatives thereof, optionally combined with conventional excipients and/or carriers.

The invention includes the use of any compounds described above containing one or more asymmetric carbon atoms which may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of formula (I) can exist in more than one tautomeric form. The invention includes methods using all such tautomers.

All terms as used herein in this specification, unless otherwise stated, shall be understood in their ordinary meaning as known in the art.

Alkyl, alkenyl, alkynyl, alkoxy, alkylthio, acyl, alkoxycarbonyl, acyloxy, acylamino, alkylsulfonyl and all other alkyl containing groups shall be understood unless otherwise specified as being C1-10, branched or unbranched where structurally possible, and optionally partially or fully halogenated. For ‘C₀₋ₙalkyl’, where n is an integer 1,2,3 etc, shall be understood to be a bond when the definition is ‘C₀’, and alkyl when n is greater than or equal to 1. Other more specific definitions are as follows:
BOC or t-BOC is tertiary-butoxycarbonyl.
t-Bu is tertiary-butyl.
DMF is dimethylformamide.
EtOAc is ethyl acetate.
EtOH and MeOH are ethanol and methanol, respectively.
TFA is trifluoroacetic acid.
THF is tetrahydrofuran.
DMSO is dimethylsulfoxide.
TBTU is O-(1 H-benzotriazol-1-yl)-N,N.N′,N′-tetramethyluronium tetrafluoroborate.
FMOC is 9-fluorenylmethoxycarbonyl.

The term “aroyl” as used in the present specification shall be understood to mean “benzoyl” or “naphthoyl”.

The term “carbocycle” shall be understood to mean an aliphatic hydrocarbon radical containing from three to twelve carbon atoms. Carbocycles include hydrocarbon rings containing from three to ten carbon atoms. These carbocycles may be either aromatic and non-aromatic ring systems, and optionally or fully halogenated. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms selected from nitrogen, oxygen and sulfur. The heterocycle may be attached by any atom of the cycle, which results in the creation of a stable structure. Unless otherwise stated, heterocycles include but are not limited to, pyrrolidinyl, morpholinyl, thiomorpholinyl, dioxalanyl, piperidinyl, piperazinyl, aziridinyl and tetrahydrofuranyl.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include but are not limited to thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl.

The term “heteroatom” as used herein shall be understood to mean atoms other than carbon such as O, N, S and P.

In all alkyl groups or carbon chains within cycloalkyl groups, where one or more carbon atoms are optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain.

Substitution on a carbon such as a methylene carbon by groups such as oxo result in definitions such as: alkoxycarbonyl, acyl, and amido, or if substituted on a ring can, for example, replace a methylene group —CH₂— with a carbonyl >C═O.

The term “aryl” as used herein shall be understood to mean aromatic carbocycle or heteroaryl as defined herein. Each aryl or heteroaryl unless otherwise specified includes its partially or fully hydrogenated derivative. For example, quinolinyl may include decahydroquinolinyl and tetrahydroquinolinyl, naphthyl may include its hydrogenated derivatives such as tetrahydranaphthyl. Each may be partially or fully halogenated. Other partially or fully hydrogenated derivatives of the aryl and heteroaryl compounds described herein will be apparent to one of ordinary skill in the art.

Terms which are analogs of the above cyclic moieties such as aryloxy or heteroaryl amine shall be understood to mean an aryl, heteroaryl, heterocycle as defined above attached to it's respective functional group.

As used herein, “nitrogen” and “sulfur” include any oxidized form of nitrogen and sulfur and the quaternized form of any basic nitrogen. For example, for an alkylthio radical such as —S—C₁₋₆ alkyl, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine. The definitions “partially or fully halogenated” “substituted by one or more halogen atoms” includes for example, mono, di or tri halo derivatives on one or more carbon atoms. A non-limiting example would be a halogenated alkyl such as —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The term “patient” refers to a warm-blooded mammal and preferably, a human.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed herein above, thereby imparting the desired pharmacological effect.

### Methods of Therapeutic Use

The compounds of the invention are effective inhibitors of Tec kinase family activity, especially of Itk. Therefore, in one embodiment of the invention, there is provided methods of treating immunological disorders using compounds of the invention. In another embodiment, there is provided methods of treating inflammatory disorders using compounds of the invention. In yet another embodiment, there is provided methods of treating allergic disorders using compounds of the invention. In yet still another embodiment, there is provided methods of enhancing memory cell generation for vaccines using compounds of the invention. In a further embodiment, there is provided methods of treating cell proliferative disorders using compounds of the invention.

Without wishing to be bound by theory, the compounds of this invention modulate T cell and mast cell activation via effective inhibition of Itk. The inhibition of T cell activation is therapeutically useful for selectively suppressing immune function. Thus, the inhibition of Itk is an attractive means for preventing and treating a variety of immune disorders, including inflammatory diseases, autoimmune diseases, organ and bone marrow transplant rejection and other disorders associated with T cell mediated immune response.

In particular, the compounds of the invention may be used to prevent or treat acute or chronic inflammation, allergies, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection) and lupus erythematosus.

The compounds of the invention are also effective inhibitors of Tec family kinases other than Itk including Txk, Tec, Btk, and Bmx and would thus be useful in treating diseases associated with the activity of one or more of these Tec family kinases.

Inhibitors of mast cell activation and degranulation block the release of allergic and pro-inflammatory mediators and cytokines. Thus inhibitors of Itk have potential utility in treating inflammatory and allergic disorders, including asthma, chronic obstructive pulmonary disease (COPD), adult respiratory distress syndrome (ARDS), bronchitis, conjunctivitis, dermatitis and allergic rhinitis. Other disorders associated with T cell or mast cell mediated immune response will be evident to those of ordinary skill in the art and can also be treated with the compounds and compositions of this invention.

Inhibitors of Itk and other Tec family kinases have potential utility in combination with other therapies for the treatment of immune, inflammatory, proliferative, and allergic disorders. Examples, though not all encompassing, include co-administration with steroids, leukotriene antagonists, anti-histamines, cyclosporin, or rapamycin.

One strategy to improve vaccination methods is to increase the number of memory T cells generated. As described in the Background, in the absence of Itk in mice, increased numbers of memory cells are generated. Thus, within the scope of the invention is the use of the present compounds in the formulation of improved vaccines that generate increased numbers of memory T cells.

For therapeutic use, the compounds of the invention may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the compounds of this invention include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

### Biological Activity

### Tec Family Kinase Assay

Itk, Txk, Tec, Btk, and Bmx are purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu₄: Tyr₁ (PGTYR). The screen is run on the Zymark Allegro robot system to dispense reagents, buffers and samples for assay, and also to wash and read plates. The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test samples initially dissolved in DMSO at 1 mg/mL, are pre-diluted for dose response (10 doses with starting final concentration of 3 μg/mL, 1 to 3 serial dilutions) with the assay buffer in 96-well polypropylene microtiter plates. A 50 μL volume/well of a mixture of substrates containing ATP (final ATP concentration in each kinase assay is equal to its apparent ATP Kₘ) and 3.6 ng/μL PGTYR-biotin (CIS Bio International) in kinase buffer is added to neutravidin coated 96-well white plate (PIERCE), followed by 25 μL/well test sample solution and 25 μL/well of diluted enzyme (1-7 nM final conc.). Background wells are incubated with buffer, rather than 25 μL enzyme. The assay plates are incubated for 30 min at room temperature. Following incubation, the assay plates are washed three times with 250 μL DELFIA wash buffer. A 100 μL aliquot of 1 nM europium-labeled anti-phosphotyrosine (Eu³⁺-PT66, Wallac CR04-100) diluted in DELFIA assay buffer is added to each well and incubated for 30 min at room temperature. Upon completion of the incubation, the plate is washed four times with 250 μL of wash buffer and 100 μL of DELFIA Enhancement Solution (Wallac) is added to each well. After 15 min of longer, time-resolved fluorescence is measured (excitation at 360 nm, emission at 620 nm) after a delay time of 250 μs.

Preferred compounds of the invention have an activity of 1 microMolar or less.

In order that this invention be more fully understood, the following examples are set forth. These examples are for the purpose of illustrating preferred embodiments of this invention, and are not to be construed as limiting the scope of the invention in any way.

The examples which follow are illustrative and, as recognized by one skilled in the art, particular reagents or conditions could be modified as needed for individual compounds without undue experimentation. Starting materials used in the schemes below are either commercially available or easily prepared from commercially available materials by those skilled in the art.

### General Synthetic Methods

The invention also provides processes for making compounds of formula I. In the scheme below, unless specified otherwise, R substituents in the formulas below shall have the meaning of R substituents in the formula I of the invention described herein above.

Intermediates used in the preparation of compounds of the invention are either commercially available or readily prepared by methods known to those skilled in the art. Reference in this regard may be made to U.S. patent application Ser. Nos. 10/288,362 and 10/632,888.

Compounds of formula I may be prepared as illustrated in Scheme I below.

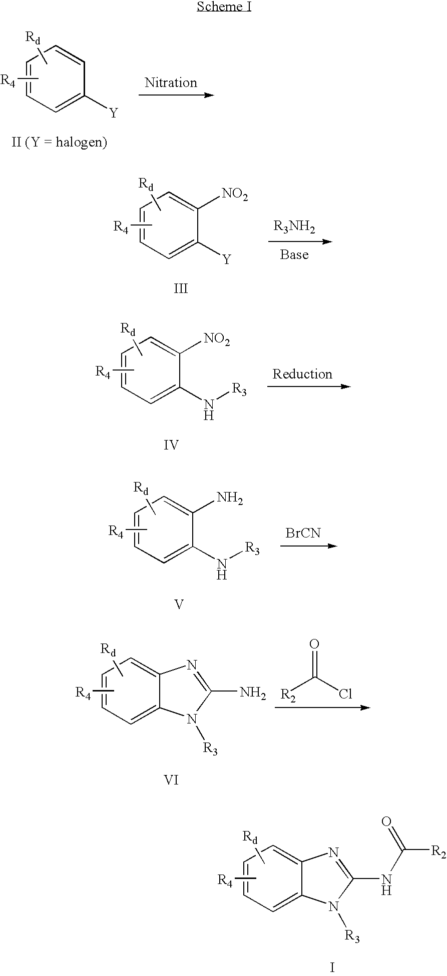

As illustrated in Scheme I, a substituted halobenzene II, preferably a substituted fluorobenzene, is nitrated by methods known in the art, for example by treatment with concentrated nitric acid and concentrated sulfuric acid to provide intermediate III. This intermediate is then reacted with R₃NH₂ in the presence of a base such as triethylamine to form IV. Reduction of the nitro group by methods known in the art, for example by treatment with hydrogen or a hydrogen source such as ammonium carbonate in the presence of a catalyst such as palladium on carbon provides V. Reaction of V with cyanogen bromide in a suitable solvent such as ethanol provides benzimidazole VI. Reaction of VI with R₂C(O)Cl in the presence of a base such as pyridine provides the desired compound of formula (I). Intermediates II may be purchased commercially or prepared by methods known in the art and illustrated in the synthetic examples below.

### SYNTHETIC EXAMPLES

### Example 1

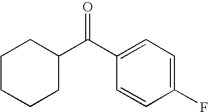

At 0° C. AlCl₃ (17.6 g, 132 mmol) was added in small portions to a solution of fluorobenzene (19.2 mL, 204 mmol) and cyclohexanecarbonyl chloride (15 g, 102 mmol) in CH₂Cl₂. After addition the reaction mixture was stirred at room temperature overnight and then was heated at 50° C. for 2 h. The reaction mixture was poured into ice and extracted with CH₂Cl₂. The combined extracts were washed with H₂O, NaHCO₃, and brine. Solvent removal and purification by flash column chromatography gave the cyclohexyl-(4-fluorophenyl)-methanone (10.5 g, 51%) as clear oil. ¹H-NMR (400 MHz, CDCl₃): δ 8.02-7.96 (m, 2 H), 7.18-7.1 (m, 2 H), 3.28-3.2 (m, 1 H), 1.96-1.25 (m, 10 H).

### Example 2

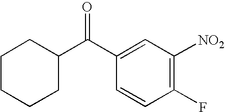

At −10° C. cyclohexyl-(4-fluoro-phenyl)-methanone (1.0 g) was added dropwise to a mixture of concentrated H₂SO₄ and 90% HNO₃ (4:4 mL). After addition the reaction mixture was stirred at −5 to −10° C. for 1.5 h. The reaction mixture was then poured into crushed ice and extracted with CH₂Cl₂. The combined extracts were washed with water, brine, and dried (Na₂SO₄). Purification by flash column chromatography gave cyclohexyl-(4-fluoro-3-nitrophenyl)-methanone (0.63 g, 52%). ¹H-NMR (400 MHz, CDCl₃): δ 8.6 (dd, 1 H), 8.22 (m, 1 H), 7.4 (dd, 1 H), 3.2 (m, 1 H), 1.9-1.2 (m, 10 H).

### Example 3

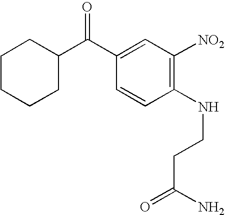

A mixture of the cyclohexyl-(4-fluoro-3-nitrophenyl)-methanone (79 mg, 0.31 mmol), H-beta-Ala-NH₂ hydrochloride (78 mg, 0.63 mmol), and N,N-diisopropyl-N′-ethylamine (DIEA) (0.35 mL) in CH₃CN (2.0 mL) was stirred at room temperature for 16 h. The reaction mixture was diluted with CH₂Cl₂ and washed with 1 N HCl, saturated NaHCO₃, brine, and dried over MgSO₄. Solvent removal gave the 3-(4-cyclohexanecarbonyl-2-nitro-phenylamino)-propionamide (98 mg, 99%). LCMS: M+H, 320.15; ¹H-NMR (400 MHz, CDCl₃): δ 8.8 (d, 1 H), 8.61 (br, 1 H), 8.1 (dd, 1 H), 7.0 (d, 1 H), 5.6 (br, 2 H), 3.8 (qt, 2 H), 3.2 (m, 1 H), 2.1 (t, 2 H), 1.9-1.2 (m, 10 H).

### Example 4

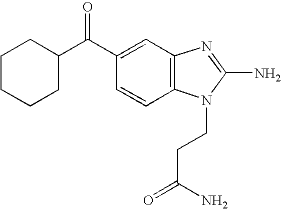

Compound 3-(4-cyclohexanecarbonyl-2-nitro-phenylamino)-propionamide (0.3 mmol) in ethanol (15 mL) was hydrogenated in the presence of Pd/C for 24 h. The reaction mixture was then filtered and concentrated. The residue was re-dissolved in ethanol (1 mL) and treated with cyanogen bromide (54 mg, 0.51 mmol) at room temperature for 2 days. Concentration and purification by column chromatography gave the 3-(2-amino-5-cyclohexanecarbonyl-benzoimidazol-1-yl)-propionamide (56 mg, 60%, 2-step). LCMS: M+H, 315.25.

### Example 5

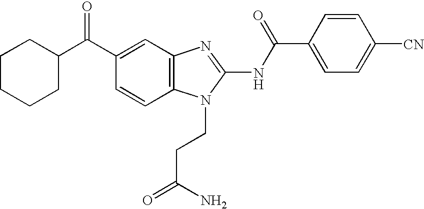

A mixture of the 3-(2-amino-5-cyclohexanecarbonyl-benzoimidazol-1-yl)-propionamide (19 mg, 0.06 mmol) and 4-cyanobenzoyl chloride (20 mg, 0.12 mmol) in CH₂Cl₂ (1 mL) and pyridine (0.5 mL) was stirred at 0° C. for 2 h, and then at room temperature for 18 h. The reaction mixture was concentrated and the residue was purified by column chromatography to give the product N-[1-(2-carbamoyl-ethyl)-5-cyclohexanecarbonyl-1H-benzoimidazol-2-yl]-4-cyano-benzamide (22 mg, 82%) LCMS: M+H, 444.21, ¹H-NMR (400 MHz, CDCl₃+DMSO-d6): δ 12.4 (m, 1 H), 8.3 (d, 2 H), 7.9 (s, 1 H), 7.8 (dd, 1 H), 7.6 (d, 2 H), 7.4 (d, 1 H), 6.6 (br, 1 H), 5.6 (br, 1 H), 4.4 (t, 2 H), 3.2 (m, 1 H), 2.78 (t, 2 H), 1.8-1.2 (m, 10 H).

### Example 6

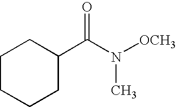

At room temperature cyclohexanecarbonyl chloride (7.5 g, 0.049 mol) was added in small portions to a mixture of N,O-dimethyl hydroxylamine hydrochloride (4.0 g, 0.041 mol) and triethyl amine (25 mL) in dry CH₂Cl₂ (40 mL). The reaction mixture was stirred at room temperature overnight. The reaction mixture was cooled to 0° C. and water was added. The organic layer was separated and washed with 1 N HCl, H₂O, 5% NaHCO₃, brine, and dried. Removal of the solvent gave the cyclohexanecarboxylic acid methoxy-methyl-amide (6.2 g, 91%). LCMS: M+H, 172.1.

### Example 7

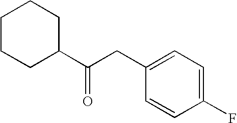

At room temperature, 4-fluorobenzyl bromide (2.5 g, 13.2 mmol) in dry ether (5 mL) was added dropwise to magnesium turnings (330 mmg, 19.59 mmol) in dry ether (10 mL). The resulting mixture was stirred at room temperature for 1 h, and heated at reflux for 1 h. The reaction mixture was cooled to 0° C. and a solution of the cyclohexanecarboxylic acid methoxy-methyl-amide (2.2 g, 13.0 mmol) in ether (5 mL) was added dropwise. The resulting mixture was allowed to warm up to room temperature in two hours and stirred at room temperature overnight. The reaction was quenched with saturated NH₄Cl, and extracted with ether. The combined extracts were concentrated, and the residue was purified by flash column chromatography to give 1-cyclohexyl-2-(4-fluoro-phenyl)-ethanone (1.6 g, 55%). ¹H-NMR (400 MHz, CDCl₃): δ 7.2 (m, 2 H) 7.0 (m, 2 H), 3.8 (s, 2 H), 2.5 (m, 1 H), 1.8-1.2 (m, 10 H).

### Example 8

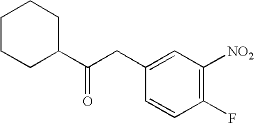

At −30° C., 1-cyclohexyl-2-(4-fluoro-phenyl)-ethanone (0.5 g) was added dropwise to a mixture of concentrated H₂SO₄ and 90% HNO₃ (2:2 mL). The mixture was stirred at −30° C. for 20-30 min. The mixture was poured into ice-H₂O and extracted with CH₂Cl₂. The combined extracts were washed with water, saturated NaHCO₃, brine, and dried (Na₂SO₄). The solvent was removed and the resulting residue was purified by flash column chromatography to give the 1-cyclohexyl-2-(4-fluoro-3-nitro-phenyl)-ethanone (140 mg, 23% ). ¹H-NMR (400 MHz, CDCl₃): δ 7.9 (dd, 7.0, 2.3 Hz, 1 H) 7.45 (m, 1 H), 7.25 (dd, J=10.6, 8.52 Hz, 1 H), 3.8 (s, 2 H), 2.5 (m, 1 H), 1.95-1.15 (m, 10 H).

### Example 9

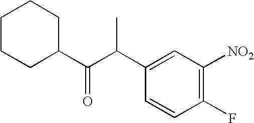

At 0° C. a solution of the 1-cyclohexyl-2-(4-fluoro-3-nitro-phenyl)-ethanone (265 mg, 1 mmol) in THF (3 mL) was added to a mixture of NaH (44 mg, 60%, 1.1 mmol) in dry THF (5.0 mL). MeI (125 □L, 2 mmol) was added and the resulting mixture was stirred at room temperature for 2 h. Water was added to the reaction mixture and the mixture was extracted with ether. The combined extracts were washed with 1 N HCl, saturated NaHCO₃, brine and dried. Solvent removal gave 1-cyclohexyl-2-(4-fluoro-3-nitro-phenyl)-propan-1-one (251 mg), which was used in the next step without further purification. ¹H-NMR (400 MHz, CDCl₃): δ 8.0 (dd, 1 H), 7.6 (m, 1 H), 7.3 (dd, 1 H), 4.0 (qt, 1 H), 2.4 (m, 1 H), 1.9-1.0 (m, 13 H).

### Example 10

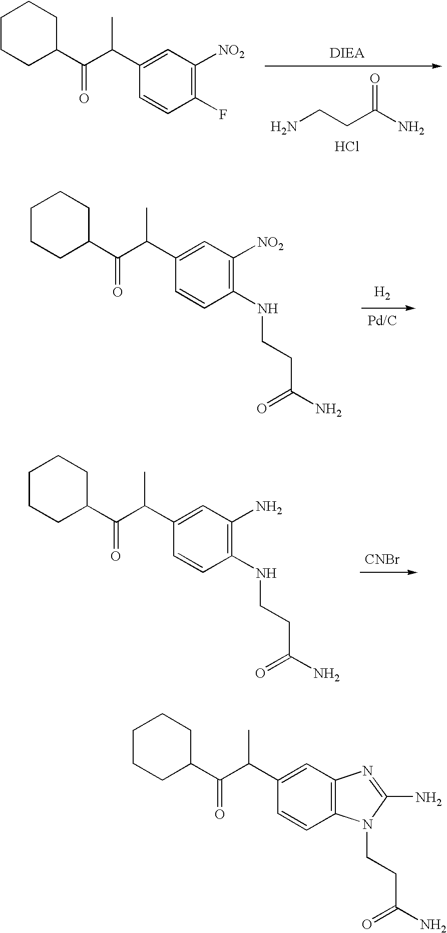

A solution of the 1-cyclohexyl-2-(4-fluoro-3-nitro-phenyl)-propan-1-one (0.45 mmol), H-□-Ala-NH₂ hydrochloride (224 mg, 1.8 mmol), and DIEA (1 mL, 5.4 mmol) in CH₃CN (5.0 mL) was heated at 70° C. for 36 h. The reaction mixture was cooled to room temperature and diluted with ether and washed with 1 N HCl, H₂O, NaHCO₃, and brine. The solution was dried and concentrated to give the desired substituted nitroaniline intermediate as a yellow solid. The solid was dissolved in ethanol (20 mL) and was hydrogenated in the presence of 10% palladium on carbon (100 mg) for 24 h. The reaction mixture was filtered and concentrated, providing the desired substituted diaminobenzene intermediate. The residue obtained was re-dissolved in ethanol (10 mL) and treated with cyanogen bromide (64 mg, 0.6 mmol) at room temperature for 36 h. Solvent removal followed by flash column chromatography gave the 3-[2-amino-5-(2-cyclohexyl-1-methyl-2-oxo-ethyl)-benzoimidazol-1-yl]-propionamide as a clear oil (45 mg, 33%, 3-step). LCMS: M+H, 343.3.

### Example 11

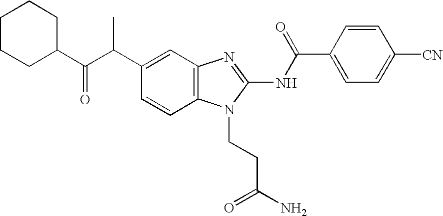

Para-cyanobenzoyl chloride (11 mg, 0.066 mmol) was added to a solution of the 3-[2-amino-5-(2-cyclohexyl-1-methyl-2-oxo-ethyl)-benzoimidazol-1-yl]-propionamide (15 mg, 0.044 mmol) in CH₂Cl₂ (2 mL) and pyridine (1 mL). The resulting mixture was stirred at room temperature for 2 days. The reaction mixture was then concentrated and the residue was purified by flash column chromatography to give the desired product, N-[1-(2-Carbamoyl-ethyl)-5-(2-cyclohexyl-1-methyl-2-oxo-ethyl)-1H-benzoimidazol-2-yl]-4-cyano-benzamide (10.6 mg, 53%). LCMS: M+H, 472.24; ¹H-NMR (400 MHz, CDCl₃): δ 8.45 (d, 2 H), 7.7 (m, 2 H), 7.4 (d, 1 H), 7.2 (t, 2 H), 6.0 (br, 1 H), 5.7 (br, 1 H), 4.5 (t, 2 H), 4.0 (qt, 1 H), 2.9 (m, 2 H), 2.5 (m, 1 H), 2.0-1.1 (m, 13 H).

### Example 12

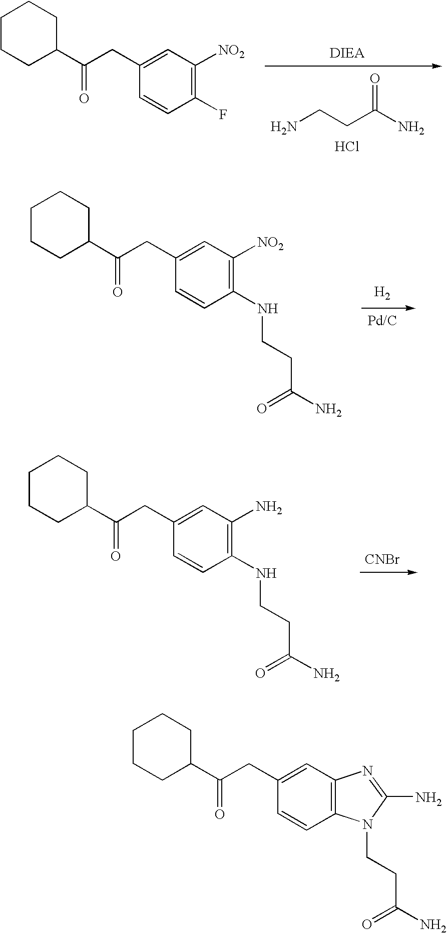

A mixture of the 1-cyclohexyl-2-(4-fluoro-3-nitro-phenyl)-ethanone (133 mg, 0.50 mmol), H-beta-Ala-NH₂ hydrochloride (248 mg, 2 mmol) and DIEA (1.2 mL, 6 mmol) in CH₃CN was heated at 50-70° C. for 3 days. The reaction mixture was then concentrated and the residue was dissolved in CH₂Cl₂ and washed with 1 N HCl, H₂O, saturated NaHCO₃, and brine. The organic layer was dried and concentrated to give the desired substituted nitroaniline intermediate as a yellowish solid. The solid was dissolved in ethanol (20 mL) and EtOAc (5 mL) and was hydrogenated in the presence of 10% palladium on carbon (100 mg) for 2.5 days. The reaction mixture was filtered and the filtrate was concentrated, providing the desired substituted diaminobenzene intermediate. The residue was dissolved in ethanol (10 mL) and treated with cyanogens bromide (64 mg) for 18 h. Concentration gave the crude 3-[2-amino-5-(2-cyclohexyl-2-oxo-ethyl)-benzoimidazol-1-yl]-propionamide, which was used for next step without further purification. LCMS: M+1: 329.2.

### Example 13

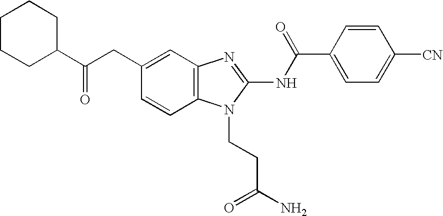

A mixture of the 3-[2-amino-5-(2-cyclohexyl-2-oxo-ethyl)-benzoimidazol-1-yl]-propionamide (0.09 mmol) and 4-cyanobenzoyl chloride (22.3 mg, 0.135 mmol) in CH₂Cl₂ (2 mL) and pyridine (1 mL) was stirred at 0° C. for 2 h, and at room temperature for 2 days. The reaction mixture was concentrated and the residue was purified by flash column chromatography to give the product, N-[1-(2-Carbamoyl-ethyl)-5-(2-cyclohexyl-2-oxo-ethyl)-1H-benzoimidazol-2-yl]-4-cyano-benzamide (14 mg). LCMS: M+1: 458.28; ¹H-NMR (400 MHz, CDCl₃+CD₃OD): δ 8.40 (d, 2 H), 7.78 (d, 2 H), 7.4 (br, 1 H), 7.2 (s, 1 H), 7.1 (d, 1 H),4.6 (t, 2 H), 3.82 (s, 2 H), 2.8 (t, 2 H), 2.45 (m, 1 H), 1.8-1.6 (m, 5 H), 1.4-1.1 (5 H).

All patents, patent applications and publications, and all literature citations in this application are fully incorporated herein by reference in their entirety.

1. A compound of the formula (I): 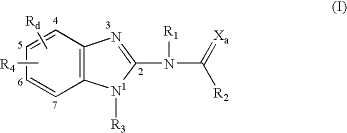
wherein:
R₁ is hydrogen or alkyl;
R₂ is chosen from aryl and heteroaryl each R₂ is optionally substituted with one or more Rₐ;
R₃ is C₁₋₁₀ alkyl chain branched or unbranched optionally substituted with one or more R_{b},
or R₃ is the group:
—(CH₂)ₙ-L-R₆, wherein L is chosen from a bond, —NH—C(O)—, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, alkyl, alkoxy, alkylthio, arylC₀₋₅ alkyl, aryloxyC₀₋₅ alkyl, heteroarylC₀₋₅ alkyl, cycloalkylC₀₋₅ alkyl, heterocyclylC₀₋₅ alkyl and amino said amino is optionally mono-or di-substituted by acyl, alkyl, alkoxycarbonyl, cycloalkylC₀₋₅ alkyl, arylC₀₋₅ alkyl, heteroarylC₀₋₅ alkyl or heterocyclylC₀₋₅ alkyl;
n is 1-10;
R₄ is a group chosen from: 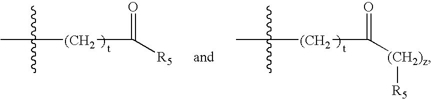
wherein a hydrogen atom for each of the —(CH₂)— groups may be replaced with a C₁₋₁₀ alkyl wherein one or more —CH₂— groups of said alkyl are optionally replaced by a heteroatom group chosen from O, S and NH, R₄ is covalently attached at the indicated 5- or 6- position of the formula (I), t and z are each independently chosen from 0, 1 or 2;
R₅ is chosen from arylC₀₋₅ alkyl, alkyl, heteroarylC₀₋₅ alkyl, cycloalkylC₀₋₅ alkyl and heterocyclylC₀₋₅ alkyl, each R₅ optionally substituted with one or more R_{c};
each Rₐ, R_{b} or R_{c} are independently chosen from hydrogen, alkyl, alkenyl, alkynyl, cycloalkyl, aryl, arylalkyl, aryloxy, alkoxy, alkylthio, acyl, alkoxycarbonyl, acyloxy, acylamino, sulphonylamino, aminosulfonyl, alkylsulfonyl, carboxy, carboxamide, oxo, hydroxy, halogen, trifluoromethyl, nitro, nitrile and amino optionally mono-or-di-substituted by alkyl, acyl or alkoxycarbonyl, wherein any of the above Rₐ, R_{b} or R_{c} are optionally halogenated where possible;
R_{d}, covalently attached at the indicated 4-, 5-, 6- or 7-position of the formula (I), is chosen from hydrogen, alkyl, alkoxy and halogen and
Xₐ and X_{b} are oxygen or sulfur;
or the pharmaceutically acceptable salts, esters, acids, isomers or tautomers thereof.

2. The compound according to claim 1 wherein:
R₁ is hydrogen;
R₂ is chosen from phenyl, naphthyl, and heteroaryl chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl and indazolyl each R₂ is optionally substituted with one or more Rₐ;
R₃ is C₁₋₁₀ alkyl chain branched or unbranched optionally substituted with one or more R_{b},
or R₃ is:
—(CH₂)ₙ-L-R₆, wherein L is chosen from a bond, —O—C(O)—, —C(O)— and —S(O)ₘ— wherein m is 0, 1 or 2, and wherein said group is optionally substituted by one or more R_{b};
wherein R₆ is independently chosen from hydrogen, hydroxy, C₁₋₅ alkyl, C₁₋₅ alkoxy, C₁₋₅ alkylthio, phenyl, naphthyl, benzyl, phenethyl, heteroarylC₀₋₅ alkyl, C₃₋₇ cycloalkylC₀₋₅ alkyl, heterocyclylC₀₋₅ alkyl and amino said amino is optionally mono-or di-substituted by C₁₋₅ acyl, C₁₋₅ alkyl, C₁₋₅ alkoxycarbonyl, arylC₀₋₅ alkyl, heteroarylC₀₋₅ alkyl or heterocyclylC₀₋₅ alkyl; and wherein each recited heteroaryl in this paragraph is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl and wherein each recited heterocyclyl in this paragraph is chosen from pyrrolidinyl, morpholinyl, thiomorpholinyl, dioxalanyl, piperidinyl and piperazinyl;
R₄ is a group chosen from: 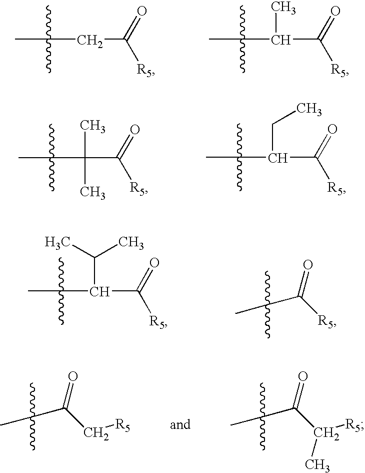
R₅ is chosen from phenyl, naphthyl, benzyl, phenethyl, C₁₋₅ alkyl, heteroarylC₀₋₅ alkyl wherein the heteroaryl is chosen from thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl and pyranyl, C₃₋₇ cycloalkylC₀₋₅ alkyl and heterocyclylC₀₋₅ alkyl wherein the heterocyclyl is chosen from aziridinyl, pyrrolidinyl, morpholinyl, thiomorpholinyl, tetrahydrofuranyl, dioxalanyl, piperidinyl and piperazinyl, each R₅ is optionally substituted with one or more R_{c};
each Rₐ, R_{b} or R_{c} are independently chosen from hydrogen, C₁₋₅ alkyl, C₂₋₅ alkenyl, C₂₋₅ alkynyl, C₃₋₈ cycloalkyl, phenyl, benzyl, phenoxy, C₁₋₅ alkoxy, C₁₋₅ alkylthio, C₁₋₅ acyl, C₁₋₅ alkoxycarbonyl, C₁₋₅ acyloxy, C₁₋₅ acylamino, C₁₋₅ sulphonylamino, aminosulfonyl, C₁₋₅ alkylsulfonyl, carboxy, carboxamide, oxo, hydroxy, halogen, trifluoromethyl, nitro, nitrile and amino optionally mono-or-di-substituted by C₁₋₅ alkyl, C₁₋₅ acyl or C₁₋₅ alkoxycarbonyl, wherein any of the above Rₐ, R_{b} or R_{c} are optionally halogenated where possible;
R_{d} is chosen from hydrogen, C₁₋₃ alkyl, C₁₋₃ alkoxy and halogen;
and
Xₐ is oxygen.

3. The compound according to claim 2 wherein:
R₂ is chosen from phenyl, naphthyl and heteroaryl chosen from thienyl, furanyl, isoxazolyl, oxazolyl, imidazolyl, thiadiazolyl, pyrazolyl, pyridinyl, quinoxalinyl and benzothienyl each R₂ is optionally substituted with one or more Rₐ;
R₆ is independently chosen from hydroxy, C₁₋₅ alkyl, C₁₋₅ alkoxy, phenyl, benzyl, phenethyl, heteroarylC₀₋₅ alkyl, heterocyclylC₀₋₅ alkyl, C₃₋₇ cycloalkyl and amino said amino is optionally mono-or di-substituted by C₁₋₅ acyl, C₁₋₅ alkyl, C₁₋₅ alkoxycarbonyl, arylC₀₋₅ alkyl or heteroarylC₀₋₅ alkyl;
and wherein each recited heteroaryl in this paragraph is chosen from thienyl, furanyl, isoxazolyl, oxazolyl, thiazolyl, thiadiazolyl, tetrazolyl, pyrazolyl, pyrrolyl and imidazolyl, each optionally substituted by R_{b};
n is 1-6;
R₅ is chosen from phenyl, naphthyl, benzyl, phenethyl, C₁₋₅ alkyl, heteroarylC₀₋₅ alkyl wherein the heteroaryl in this paragraph is chosen from thienyl, furanyl, imidazolyl and pyridinyl, C₃₋₇ cycloalkylC₀₋₅ alkyl and heterocyclylC₀₋₅ alkyl wherein the heterocyclyl is chosen from aziridinyl, pyrrolidinyl, tetrahydrofuranyl, tetrahydropyridinyl, morpholinyl, thiomorpholinyl, piperidinyl and piperazinyl, each R₅ is optionally substituted with one or more R_{c};
R_{d} is chosen from hydrogen and C₁₋₃ alkyl.

4. The compound according to claim 3 wherein:
R₂ is chosen from phenyl and heteroaryl chosen from thienyl, furanyl, isoxazolyl, thiadiazolyl, pyrazolyl and pyridinyl each R₂ is optionally substituted with one or more Rₐ;
R₃ is:
—(CH₂)ₙ—C(O)—R₆ or
—(CH₂)ₙ—R₆;
wherein R₆ is independently chosen from hydroxy, C₁₋₅ alkyl, C₁₋₅ alkoxy, phenyl, morpholinylC₀₋₅ alkyl, piperazinylC₀₋₅ alkyl, imidazolylC₀₋₅ alkyl, pyrrolidinylC₀₋₅ alkyl, pyrrolidinonylC₀₋₅ alkyl, thienylC₀₋₅ alkyl, C₃₋₇ cycloalkyl and amino said amino is optionally mono-or di-substituted by C₁₋₅ alkyl or C₁₋₅ alkoxycarbonyl;
R₅ is chosen from phenyl, furanyl, benzyl, phenethyl, C₁₋₃ alkyl and C₃₋₇ cycloalkylC₀₋₅ alkyl each optionally substituted with one or more R_{c};
each Rₐ, R_{b} or R_{c} are independently chosen from C₁₋₅ alkyl, C₃₋₈ cycloalkyl, phenyl, C₁₋₅ alkoxy, amino optionally mono-or-di-substituted by C₁₋₅ alkyl, C₁₋₅ alkoxycarbonyl, carboxamide, hydroxy, halogen, trifluoromethyl, nitro and nitrile, wherein any of the above Rₐ, R_{b} or R_{c} are optionally halogenated where possible;
and
R_{d} is chosen from hydrogen and methyl.

5. The compound according to claim 4 wherein:
R₂ is chosen from phenyl, thienyl, furanyl, isoxazolyl and pyridinyl each optionally substituted with one or more Rₐ;
R₅ is chosen from methyl, CF₃, cyclopentyl, phenyl and cyclohexyl each optionally substituted with one or more R_{c};
R_{d} is hydrogen and
n is 2-5.

6. The compound according to claim 5 wherein:
R₂ is chosen from phenyl, thien-2-yl, isoxazol-5-yl and pyridin-3-yl each optionally substituted with one or more Rₐ;
R₄ is chosen from: 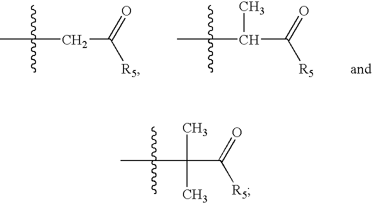
R₆ is independently chosen from hydroxy, methyl, ethyl, C₁₋₃ alkoxy, phenyl, morpholinyl, piperazinyl, imidazolyl, pyrrolidinyl, pyrrolidinonyl, thienylC₀₋₅ alkyl, C₃₋₇ cycloalkyl and amino said amino is optionally mono-or di-substituted by C₁₋₅ alkyl or C₁₋₅ alkoxycarbonyl;
and
each Rₐ, R_{b} or R_{c} are independently chosen from C₁₋₃ alkoxy, amino optionally mono-or-di-substituted by C₁₋₃ alkyl, carboxamide, hydroxy, fluoro, chloro, bromo, trifluoromethyl, nitro and nitrile.

7. The compound according to any one of claims 1-6 wherein:
R₄ is covalently attached at the indicated 5-position of the formula (I) or R₄ is covalently attached at the indicated 6-position of the formula (I).

8. A compound chosen from: 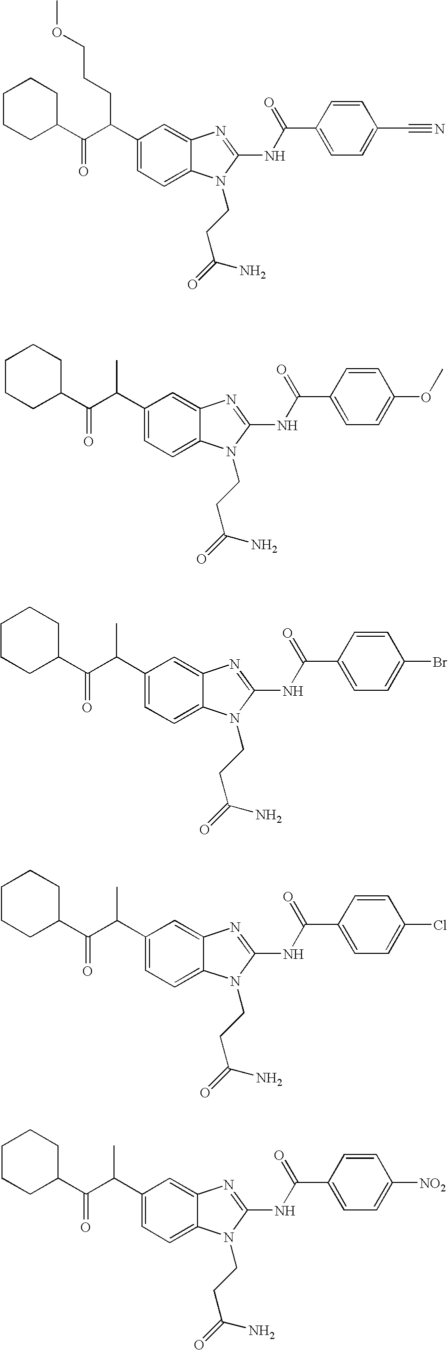 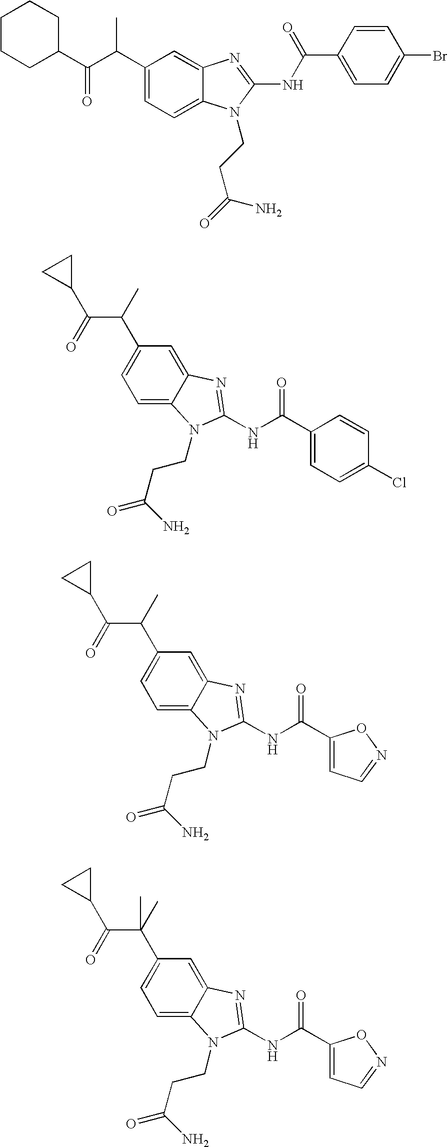 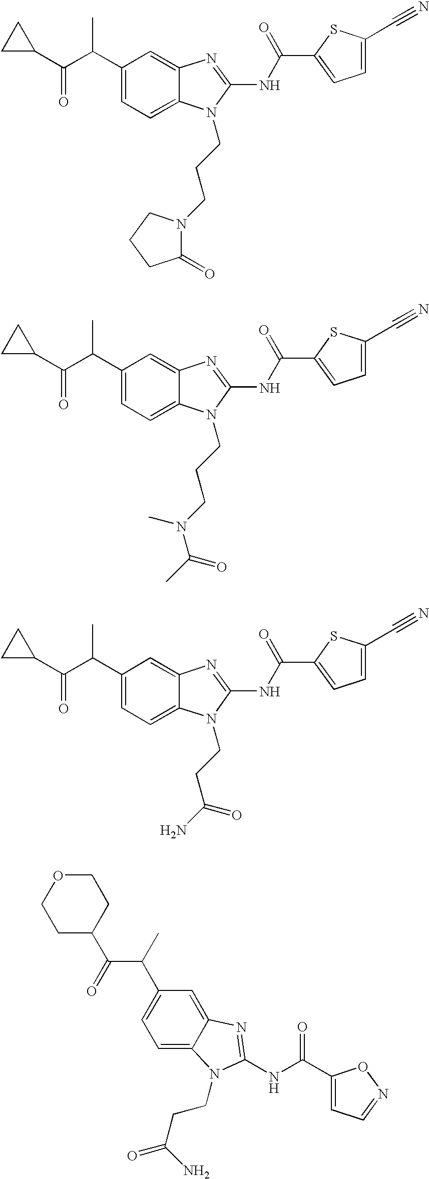 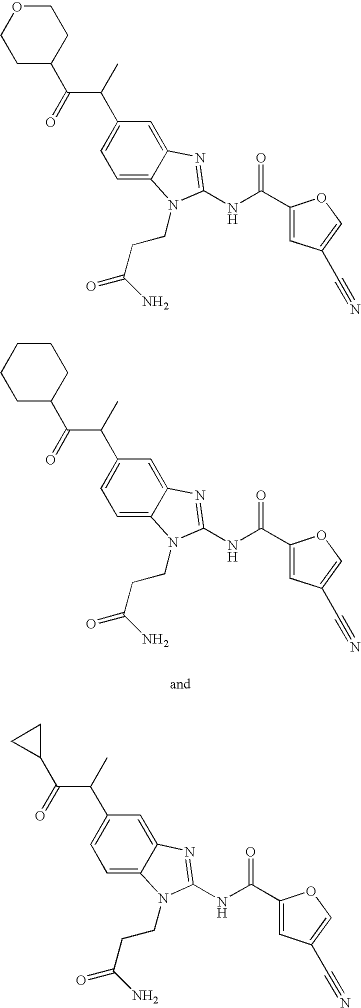
or
the pharmaceutically acceptable salts, esters, acids, isomers or tautomers thereof.

9. A compound chosen from: 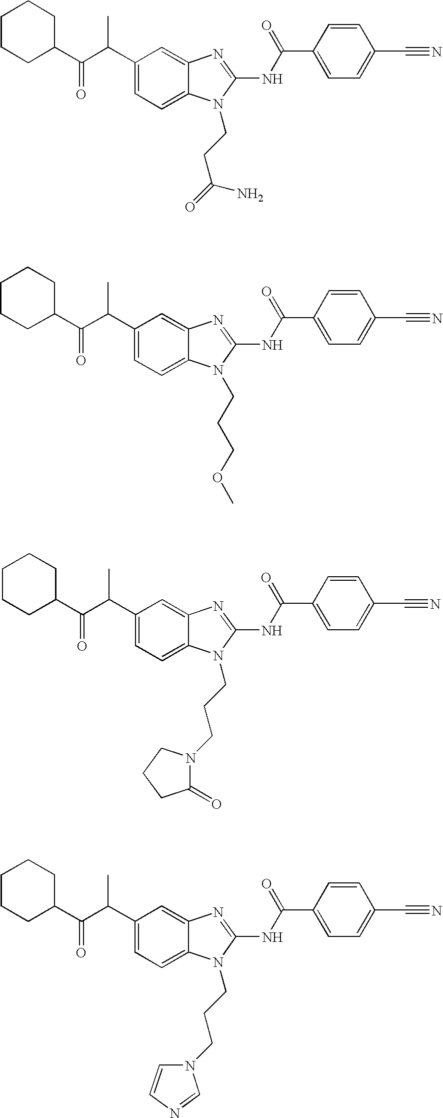 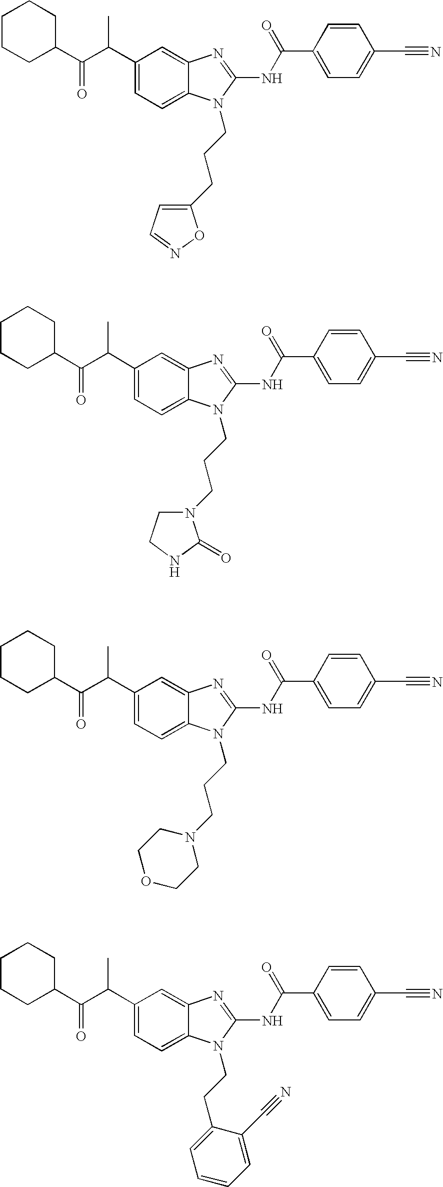 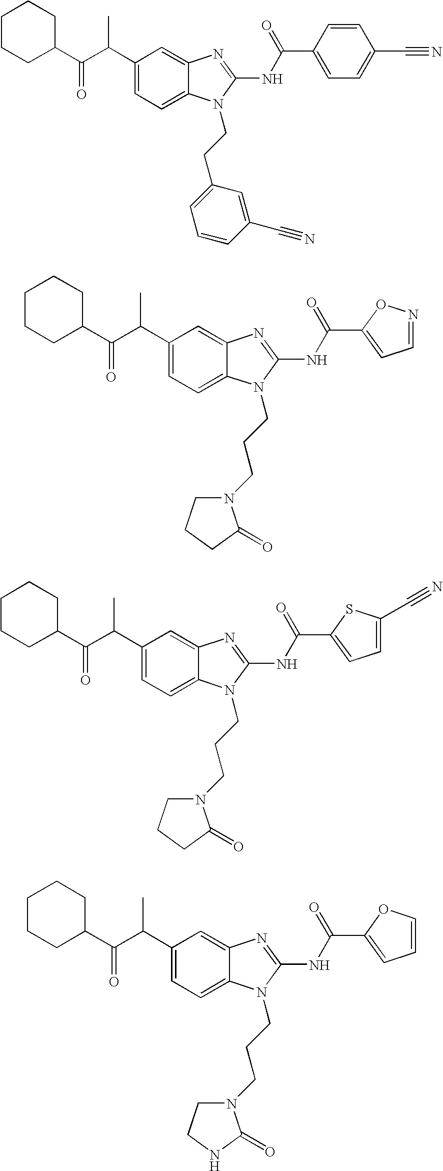 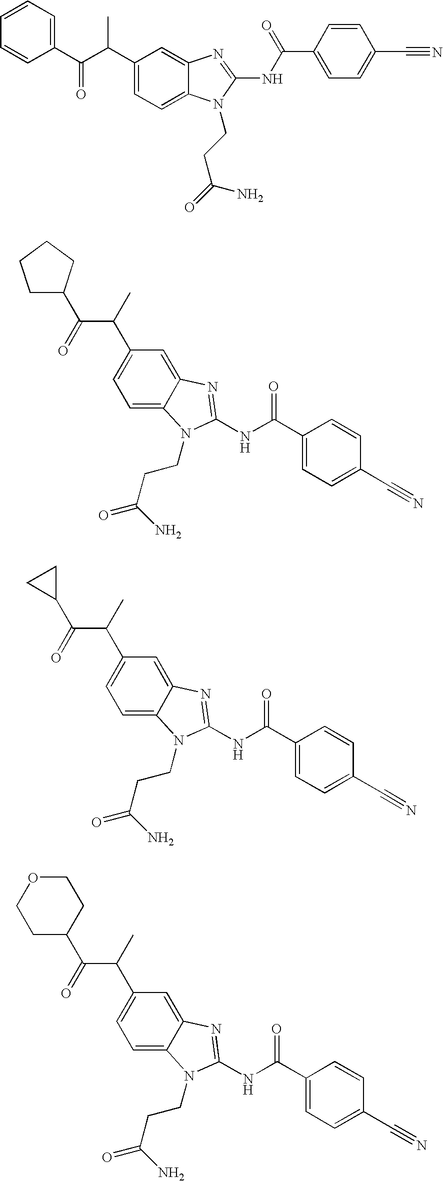 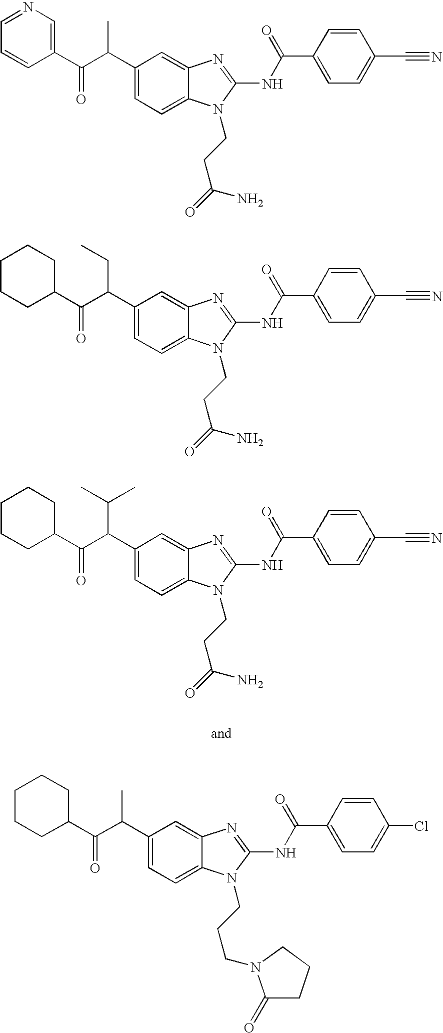
or
the pharmaceutically acceptable salts, esters, acids, isomers or tautomers thereof.

10. A pharmaceutical composition comprising a pharmaceutically effective amount of a compound according to claim 1 and one or more pharmaceutically acceptable carriers and/or adjuvants.

11. A method of treating an allergic disorder said method comprising administering to a patient in need thereof a therapeutically effect amount of a compound according to claim 1.

12. A method of treating a disease chosen from chronic inflammation, cancer, contact dermatitis, psoriasis, rheumatoid arthritis, multiple sclerosis, type 1 diabetes, inflammatory bowel disease, Guillain-Barre syndrome, Crohn's disease, ulcerative colitis, graft versus host disease, lupus erythematosus, asthma, chronic obstructive pulmonary disease (COPD), adult respiratory distress syndrome (ARDS), bronchitis, conjunctivitis, dermatitis and allergic rhinitis said method comprising administering to a patient in need thereof a therapeutically effect amount of a compound according to claim 1.
